(19) United States
(12) Patent Application Publication
(10) Pub. No.: US 2004/0039059 A1
(43) Pub. Date: Feb. 26, 2004

(54) Use of n,n' -dibenzyl ethylene diamine-n, n'- diacetic acid derivatives as anti-pollution agent

(76) Inventors: Philippe Catroux, Nogent-sur-Marne (FR); Jose Cotovio, Danmartin-en-Goele (FR); Daniel Duche, Paris (FR); Jean-Baptiste Galey, Aulnay sous Bois (FR)

Correspondence Address: D Douglas Price, Steptoe & Johnson, 1330 Connecticut Ave N W, Washington, DC, 20036, US

(21) Appl. No.: 10/276,539

(22) PCT Filed: May 14, 2001
(86) PCT No.: PCT/FR01/01452

(30) Foreign Application Priority Data
May 18, 2000 (FR) 00/06381

(51) Int. Cl. A61K 31/195

(52) U.S. Cl. 514/564

(57) ABSTRACT

The invention concerns the use for topical application of at least a N,N′-dibenzyl ethylene diamine N,N′-acetic acid derivative or salt, a metal complex or an ester of such a derivative as anti-pollution agent, preferably as cosmetic anti-pollution agent.

The present invention relates to the use in topical application of N,N′-dibenzylethylenediamine-N,N′-diacetic acid derivatives as an antipollution agent, and also to a cosmetic treatment process using said derivatives.

Metal ions are required by the body in the form of trace amounts as essential nutrients. For example, several functions involving polypeptides, such as enzymatic, structural and immunological functions, require metallic cofactors.

However, other metal ions, in particular heavy metal ions when they are at nonphysiological concentrations, may impair these functions. Thus, overexposure to metals of the environment can lead to toxic effects.

Ecological studies conducted in industrialized countries show that the amounts of metals present in the atmosphere are on the increase. This leads to an increase in the levels of heavy metals in body tissues following the ingestion of contaminated food and exposure to atmospheric metals.

The effects of accumulation of heavy metals may be extremely hazardous and their toxicity is partially due to the impairment of the tertiary and quaternary structures of proteins, which results in a reduction in their catalytic activity. The impaired proteins may become antigenic and bring about an immune response. They are then recognized by the body as foreign polypeptides and may give rise to autoimmune responses.

Another mechanism responsible for the toxic effects of metals is the competitive substitution of natural physiological cofactors with heavy metals at nonphysiological concentrations. Thus, controlling the pollutant heavy metals in the atmosphere is essential for preventing diseases in relation with exposure to metals.

Due to the increasing contamination of the environment with heavy metals and their ubiquitous presence in the ecosystem, the skin, the hair and the accessible mucous membranes represent the largest area of contact and thus promote the accumulation of metals and their subsequent absorption into the body.

Certain metals and metal compounds present in industrial manufactured products, chemicals, jewelry, clothing, medicinal products, colorants and cleaning products are involved in primary irritation reactions, allergic reactions and carcinogenicity reactions in skin tissue.

The metals that are the main offenders in the environment are copper, cobalt, zinc, manganese, mercury, nickel, lead and cadmium.

Skin rashes caused by metal-induced dermatitis are a problem encountered in people exposed to large amounts of certain metal ions. Exposure to nickel in the environment is largely due to the frequent use of this metal in jewelry articles, watch straps and clothing buttons. Sensitization to nickel with the development of dermatitis is an industrial hazard in certain occupations.

Moreover, the deposition of minerals on the hair is an inevitable phenomenon. The hair is a strong absorber of metals. The binding is so strong that once these bound metals have been captured by the anionic sites of the keratin fiber, they are difficult to elute. The degree of binding of metals to the hair generally depends on several factors such as the size of the fiber, its porosity and the exposure time. Metals such as copper, lead and iron may interfere with chemical treatments such as the dyeing and permanent waving of the hair.

Certain cosmetic products contain metals such as magnesium, copper or iron. The absorption of these metals by keratin fibers may interfere with chemical treatments such as dyeing, bleaching or permanent-waving effects. These interactions may lead to problems in dyeing or precipitations, as described in American patent U.S. Pat. No. 5,635,167.

It has been demonstrated that certain heavy metals penetrate into the skin and are accumulated (A. B. G. Landsdown. Critical Reviews in Toxicology, 1995, 25:397-462). At high concentrations, they can induce oxidation mechanisms on membrane lipids, direct cytotoxicity, liable to result in cellular necrosis, and alkylation of the cellular nucleophiles, which may be the cause of sensitization phenomena or carcinogenesis.

(S. J. Stochs and D. Bagchi. Free Radical Biology and Medicine. 1995, 18:321-336; M. E. Figueiredo Pereira et al., The Journal of Biological Chemistry. 1998, 21:12703-12709; N. L. Acan et al., 1995, Biochemical and Molecular Medicine. 54:33-37.)

Thus, there is a need for compositions for preventing the harmful effects caused by these pollutants and for protecting keratin materials.

Solutions in cosmetic and therapeutic treatments have already been envisioned. It has been found that compounds with sulfur-containing groups behave like heavy metal sequestering agents, for instance the metallothioneines in patent EP 0 557 042 A1 or the amino acid compounds with sulfur-containing groups in patent application EP 0 914 815 A1.

Patent application GB 2 333 705 A mentions the use of ethylenediaminedisuccinic acid in compositions for treating heavy-metal-induced skin irritations.

Moreover, patent application WO 94/11338 describes the use of N-arylmethyleneethylenediamine triacetate, N-arylmethylene iminodiacetate or N,N′-diarylmethylene-ethylenediamine acetate for combating oxidative stress, i.e. its therapeutic use for treating cancer, inflammatory conditions, ischemia/reinfusion and nervous system diseases, and its use for combating ageing.

More recently, N,N′-dibenzylethylenediamine-N,N′-diacetic acid derivatives have been used as skin depigmenting agents (EP 0 820 763).

The problem posed is thus that of protecting the skin against metals and the deleterious effects thereof encountered in urban pollution, by selecting active principles that are effective in controlling these effects and/or preventing the penetration of metals into the skin.

It has now been found, entirely surprisingly, that the use of N,N′-dibenzylethylenediamine-N,N′-diacetic acid derivatives allows keratin materials to be protected against the effects of pollutants.

N,N′-Dibenzylethylenediamine-N,N′-diacetic acid displays fewer side-effects than other complexing molecules, for instance deferoxamine, EHPG or DTPA. Furthermore, this molecule has antioxidant capacities.

Consequently, this molecule makes it possible to protect keratin material against the effects of pollutants and to form with iron complexes with a low association constant, which reduces the toxicological risks associated with a disruption in cell metabolism.

The topical application of the molecule in nonesterified form makes it possible to maintain the active agent in an extracellular environment, thus making it possible firstly to limit the risks of interaction with the intracellular divalent ions and secondly to avoid concentration, by chelating the metals originating from the pollution, in the cell.

These derivatives allow better cell regeneration and also a return to homeostasis, thus making it possible to obtain healthier keratin materials.

Thus the main subject of the present invention is the use in topical application of at least one N,N′-dibenzylethylenediamine-N,N′-diacetic acid derivative as an antipollution agent, preferably as an antipollution cosmetic agent.

The expression “antipollution cosmetic agent” means an agent that protects the skin and keratin materials so as to prevent, attenuate and/or eliminate the deleterious effects of metals.

These derivatives may especially correspond to those of formula (I):

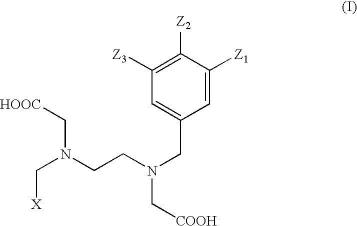

in which X represents COOH or a ring

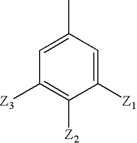

in which Z₁, Z₂ and Z₃, independently of each other, may be H, OR or R, R representing a linear or branched, saturated or unsaturated, optionally substituted C₁-C₈ alkyl radical, or a salt, a metal complex or an ester of such a derivative.

The C₁-C₈ alkyl radical is preferably a saturated C₁-C₄ radical, such as methyl, ethyl, isopropyl or tert-butyl.

Examples of possible substituents that may be mentioned include the hydroxyl group and halogens.

Salts that may be mentioned include the addition salts with a mineral or organic acid, for instance sulfuric acid, hydrochloric acid, nitric acid, phosphoric acid or acetic acid, and addition salts with a mineral or organic base, for instance sodium hydroxide, potassium hydroxide or triethanolamine.

Metal complexes that may be mentioned include the complexes formed by addition of ZnCl₂ or CaCl₂, for example.

Esters that may especially be mentioned include the methyl ester or the ethyl ester.

The compounds used in the present invention may be prepared according to the methods described in WO 94/11338.

Preferably, the derivative of formula (I) is chosen from N,N′-bis(3-hydroxybenzyl)ethylenediamine-N,N′-diacetic acid and N,N′-bis(3,4,5-trimethoxybenzyl)-ethylenediamine-N,N′-diacetic acid.

According to the invention, the derivatives of formula (I) are used in order to protect keratin materials against the effects of pollutant metals or metalloid elements.

In the context of the present invention, the expression “keratin materials” especially means the skin, the scalp, the hair, the eyelashes, the eyebrows, the nails and mucous membranes.

The derivatives of formula (I) are used for their cytoprotective effect on keratin materials with respect to pollutant metals or metalloid elements.

They are also used as antipolluting agents for increasing cell regeneration and a return to homeostasis in keratin materials, in order to obtain healthier keratin materials.

A subject of the invention is also the use of at least one derivative of formula (I) as defined above or a salt, a metal complex or an ester of such a derivative, in or for the preparation of an antipolluting composition, which is preferably cosmetic, for topical application.

It has been found that the compositions used according to the invention also have good stability properties and do not show any problems of toxicity associated with a disruption of cell metabolism.

The antipolluting composition in accordance with the invention may contain from 0.005% to 10% and preferably 0.1% to 5% by weight of compound of formula (I) relative to the total weight of the composition.

The composition used in the invention may also contain at least one other antipollution compound.

Said antipollution compound is chosen especially from anthocyans and/or derivatives thereof, compounds containing a thioether, sulfoxide or sulfone function, ergothioneine and/or its derivatives, metal-chelating agents such as antioxidants and extracts of cells of a plant of the Pontederiacea family. Among the antioxidants that will be chosen more particularly are polyphenols and, inter alia, ellagic acid.

The composition used in the invention, which is preferably a cosmetic composition, also comprises a cosmetically acceptable medium more particularly consisting of water and/or optionally of a cosmetically acceptable organic solvent. The organic solvents may represent from 5% to 98% of the total weight of the composition. They may be chosen from the group consisting of hydrophilic organic solvents, lipophilic organic solvents and amphiphilic solvents, or mixtures thereof. Among the hydrophilic organic solvents that may be mentioned, for example, are mono- or polyfunctional alcohols such as linear or branched lower monoalcohols containing from 1 to 8 carbon atoms, for instance ethanol, propanol, butanol, isopropanol and isobutanol, optionally oxyethylenated polyethylene glycols containing from 6 to 80 ethylene oxides, polyols such as propylene glycol, isoprene glycol, butylene glycol, glycerol, sorbitol and its derivatives, monoalkyl or dialkyl isosorbides, the alkyl groups of which contain from 1 to 5 carbon atoms, for instance dimethyl isosorbide, glycol ethers, for instance diethylene glycol monomethyl ether or monoethyl ether, and propylene glycol ethers, for instance dipropylene glycol methyl ether. Lipophilic organic solvents that may be mentioned, for example, include fatty esters such as diisopropyl adipate, dioctyl adipate and alkyl benzoates. Amphiphilic organic solvents that may be mentioned include polyols such as propylene glycol (PPG) derivatives, such as esters of polypropylene glycol and in particular esters of polypropylene glycol and of fatty acids, derivatives of PPG and of fatty alcohols, for instance PPG-23 oleyl ether, and PPG-36 oleate.

In order for the cosmetic or dermatological compositions of the invention to be more pleasant to use (softer to apply, more nourishing and more emollient), it is possible to add a fatty phase to the medium of these compositions.

The fatty phase preferably represents from 0 to 50% relative to the total weight of the composition.

This fatty phase may comprise one or more oils preferably chosen from the group consisting of:

volatile or nonvolatile, linear, branched or cyclic, organomodified or non-organomodified, water-soluble or liposoluble silicones,

mineral oils such as liquid paraffin and liquid petroleum jelly,

oils of animal origin such as perhydrosqualene,

oils of plant origin such as sweet almond oil, avocado oil, castor oil, olive oil, jojoba oil, sesame oil, groundnut oil, macadamia oil, grape-seed oil, rapeseed oil or coconut oil,

synthetic oils such as purcellin oil and isoparaffins,

fluoro oils and perfluoro oils,

fatty acid esters such as purcellin oil.

Said fatty phase may also comprise as fatty substances one or more fatty alcohols, fatty acids or waxes (paraffin wax, polyethylene wax, carnauba wax or beeswax).

In a known manner, all the compositions of the invention may also contain adjuvants that are common in the cosmetic and dermatological field, such as standard aqueous or lipophilic gelling agents and/or thickeners, hydrophilic or lipophilic active agents, preserving agents, antioxidants, fragrances, emulsifiers, moisturizers, pigmenting agents, depigmenting agents, keratolytic agents, vitamins, emollients, sequestering agents, surfactants, polymers, acidifying or basifying agents, fillers, free-radical scavengers, ceramides, sunscreens, especially ultraviolet screening agents, insect repellents, slimming agents, colorants, bactericides and antidandruff agents.

The amounts of these various adjuvants are those conventionally used in the fields under consideration.

Needless to say, a person skilled in the art will take care to select the optional compound(s) to be added to the composition according to the invention, such that the advantageous properties intrinsically associated with the composition in accordance with the invention are not, or are not substantially, adversely affected by the envisioned addition.

The compositions according to the invention may be in any presentation form normally used for topical application, especially in the form of an aqueous, aqueous-alcoholic or oily solution, an oil-in-water or water-in-oil or multiple emulsion, an aqueous or oily gel, a liquid, pasty or solid anhydrous product or a dispersion of oil in an aqueous phase with the aid of spherules, these spherules possibly being polymer nanoparticles such as nanospheres and nanocapsules, or better still lipid vesicles of ionic and/or nonionic type.

The compositions according to the invention may be more or less fluid and may have the appearance of a white or colored cream, an ointment, a milk, a lotion, a serum, a paste or a mousse.

They may optionally be applied to the skin in aerosol form.

They may also be applied in solid form, and for example in the form of a stick.

They may be used as care products and/or as makeup products.

The compositions of the invention may have a pH of between 3 and 8 and preferably between 5 and 7.

Another subject of the invention consists of a cosmetic treatment process for protecting the body against the effects of pollution, which consists in applying to keratin material a cosmetically effective amount of at least one derivative of formula (I) as defined above.

Another cosmetic treatment process according to the invention, for protecting the body against the effects of pollution, consists in applying to keratin material a cosmetic composition according to the invention as defined above.

The examples that follow are intended to illustrate the invention without, however, being limiting in nature.

### EXAMPLE I

Protective effect of N,N′-bis(3,4,5-trimethoxybenzyl)-ethylenediamine-N,N′-diacetic acid

I Protocol

The cytotoxic effect of cadmium on human keratinocytes in culture was evaluated by means of a technique of measuring the cell viability, known as the neutral red incorporation test (E. Borenfreund and J. A. Puerner, Tissue Culture Methods. 1984, 9: 7-9).

The study was performed on a monolayer culture of human keratinocytes obtained from plastic surgeries. The cells are inoculated on D-3 into 96-well culture dishes at a rate of 25 000 cells/cm² in 100 μl of culture medium (calf serum-free defined medium, NR2, Biofluids). The incubations are performed in an incubator at 37° C. under a humid atmosphere enriched with 5% CO₂.

The cells are treated for 24 hours with increasing concentrations (0, 10, 25, 50, 75, 100, 150 and 200 μM) of cadmium chloride (CdCl₂) alone, so as to determine its cytotoxicity. In parallel, a treatment is performed under the same conditions, but in the presence of N,N′-bis(3,4,5-trimethoxybenzyl)ethylenediamine-N,N′-diacetic acid (100 and 50 μm). The incubations are performed in an incubator at 37° C., [lacuna] % CO₂ under a humid atmosphere.

At the end of the period of contact, a neutral red incorporation test is performed and the results are read on a spectrophotometer at 550 nm.

To do this, the cells are rinsed with PBS buffer in order to remove the treatment solutions; 100 μl per well of a 0.5 mg/ml neutral red solution are added to the culture medium; the mixture is incubated at 37° C. and 5% CO₂, under a humid atmosphere, for three hours; the resulting mixture is rinsed with PBS; fixing is performed with formaldehyde/calcium solution for one minute; the neutral red is extracted with 100 μl/well of ethanol-acetic acid solution; the optical density is read on the spectrophotometer at 550 nm and the CdCl₂ concentration, resulting in a 50% reduction in viability=IC-50, is calculated.

II. Results:

The results are average results. Three independent experiments were performed and four measurements were taken per experiment.

The results, given as the concentration resulting in a 50% drop in cell viability, are collated in the following table:

Cytotoxicity of cadmium chloride toward human keratinocytes in culture, in the presence or absence of N,N′-dimethylethylenediamine-N,N′-diacetic acid, at two concentrations: 50 μM and 100 μM.

IC.50 of cadmium chloride

| without protecting agent | with protecting agent | |
|---|---|---|
| Mean ± SEM | Mean ± SEM | |
| 39 ± 1.15 μM | 50 μM | 100 μM |
| | 92 ± 7.5 μM | 122 ± 4.6 μM |

Cadmium chloride alone shows considerable toxicity, with an IC.50 of 39 μM. In the presence of N,N′-dimethylethylenediamine-N,N′-diacetic acid, the cytotoxicity of cadmium chloride decreases significantly (which corresponds to an increase in the IC.50).

at 50 μM of protecting agent, the cytotoxicity decreases by a factor of 2.3.

at 100 μM of protecting agent, the cytotoxicity decreases by a factor of 3.1.

III. Conclusion

Starting with an in vitro biological model it has been shown that a representative agent of a category of atmospheric pollutants such as cadmium leads under the experimental conditions to strong toxicity, and that N,N′-bis (3,4,5-trimethoxybenzyl)ethylenediamine-N,N′-diacetic acid significantly protects the cells against the toxicity of this pollutant.

### FORMULATION EXAMPLES

### Example 1

According to the usual preparation techniques, the constituents below are mixed together to prepare an emulsion.

| | | |
|---|---|---|
| N,N′-bis (3,4,5-trimethoxybenzyl)- | 1 | g |
| ethylenediamine-N, N′-diacetic acid | | |
| polyethylene glycol oxyethylenated | 3 | g |
| with 50 mol of ethylene oxide | | |
| monodiglyceryl stearate | 3 | g |
| liquid petroleum jelly | 24 | g |
| cetyl alcohol | 5 | g |
| water qs | 100 | g |

### Example 2

In the same manner, an emulsion is prepared according to a standard technique, using the following compounds:

| | | |
|---|---|---|
| N,N′-bis (3-hydroxybenzyl) ethylene- | 0.1 | g |
| diamine-N, N′-diacetic acid | | |
| octylpalmitate | 10 | g |
| glyceryl isostearate | 4 | g |
| liquid paraffin | 24 | g |
| vitamin E | 1 | g |
| glycerol | 3 | g |
| water qs | 100 | g |

### Example 3

Starting with the constituents below, the following composition is formulated:

| | | |
|---|---|---|
| N,N′-bis (3-hydroxybenzyl) ethylene- | 2 | g |
| diamine-N,N′-diacetic acid | | |
| jojoba oil | 13 | g |
| methyl isopropyl para-benzoxybenzoate | 0.05 | g |
| potassium sorbate | 0.3 | g |
| cyclopentadimethylsiloxane | 10 | g |
| stearyl alcool | 1 | g |
| stearic acid | 4 | g |
| polyethylene glycol stearate | 3 | g |
| vitamin E | 1 | g |
| glycerol | 3 | g |
| water qs | 100 | g |

### Example 4

Starting with the constituents below, the following composition is formulated:

| | | |
|---|---|---|
| N,N′-bis (3-hydroxybenzyl)ethylene- | 1 | g |
| diamine-N,N′-diacetic acid | | |
| liquid paraffin | 13 | g |
| methyl isopropyl para-benzoxybenzoate | 0.05 | g |
| potassium sorbate | 0.3 | g |
| cyclopentadimethylsiloxane | 10 | g |
| ellagic acid | 0.1 | g |
| stearic acid | 4 | g |
| polyethylene glycol stearate | 3 | g |
| vitamin E | 1 | g |
| glycerol | 3 | g |
| water qs | 100 | g |

1. The use in topical application as an antipollution agent, preferably as an antipollution cosmetic agent, of at least one derivative of formula (I): 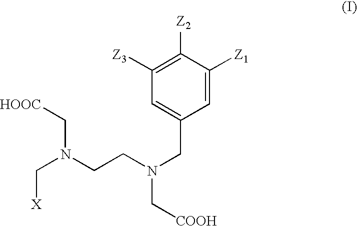
in which X represents COOH or a ring 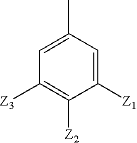
in which Z₁, Z₂ and Z₃, independently of each other, may be H, OR or R, R representing a linear or branched, saturated or unsaturated, optionally substituted C₁-C₈ alkyl radical, or a salt, a metal complex or an ester of such a derivative.

2. The use in topical application of at least one derivative of formula (I) as defined in claim 1, as an agent, preferably a cosmetic agent, with a cytoprotective effect on keratin materials with respect to pollutant elements or metalloid elements.

3. The use in topical application of at least one derivative of formula (I) as defined in claim 1 or 2, for increasing cell regeneration and a return to homeostasis in keratin materials, in order to obtain healthier keratin materials.

4. The use as claimed in any one of claims 1 to 3, characterized in that the derivative of formula (I) is chosen from N,N′-bis(3-hydroxybenzyl)ethylenediamine-N,N′-diacetic acid and N,N′-bis(3,4,5-trimethoxybenzyl)ethylenediamine-N,N′-diacetic acid.

5. The use of a derivative of formula (I) as defined according to any one of claims 1 to 4, in or for the preparation of an antipolluting composition, which is preferably cosmetic, for topical application.

6. The use as claimed in claim 5, characterized in that said antipolluting cosmetic composition contains from 0.005% to 10% and preferably 0.1% to 5% by weight of compound of formula (I) relative to the total weight of the composition.

7. The use as claimed in claim 5 or 6, characterized in that said composition also contains at least one other antipollution compound.

8. The use as claimed in claim 7, characterized in that said antipollution compound is chosen from anthocyans and/or derivatives thereof, compounds containing a thioether, sulfoxide or sulfone function, ergothioneine and/or its derivatives, metal-chelating agents such as antioxidants and in particular ellagic acid, and extracts of cells of a plant of the Pontederiacea family.

9. The use as claimed in any one of claims 5 to 8, characterized in that the composition also comprises a cosmetically acceptable medium consisting of water and/or of at least one organic solvent chosen from the group consisting of hydrophilic organic solvents, lipophilic organic solvents and amphiphilic solvents, or mixtures thereof.

10. The use as claimed in claim 9, characterized in that the organic solvents are chosen from the group consisting of mono- or polyfunctional alcohols, optionally oxyethylenated polyethylene glycols, polypropylene glycol esters, sorbitol and its derivatives, dialkyl isosorbides, glycol ethers and polypropylene glycol ethers, and fatty esters.

11. The use as claimed in claim 9 or 10, characterized in that the organic solvent(s) represent(s) from 5% to 98% relative to the total weight of the composition.

12. The use as claimed in any one of claims 5 to 11, characterized in that the composition also comprises at least one fatty phase.

13. The use as claimed in claim 12, characterized in that the fatty phase represents from 0 to 50% relative to the total weight of the composition.

14. The use as claimed in any one of claims 5 to 13, characterized in that the composition also contains at least one additive chosen from the group consisting of standard aqueous or lipophilic gelling agents and/or thickeners, hydrophilic or lipophilic active agents, preserving agents, antioxidants, fragrances, emulsifiers, moisturizers, pigmenting agents, depigmenting agents, keratolytic agents, vitamins, emollients, sequestering agents, surfactants, polymers, acidifying or basifying agents, fillers, free-radical scavengers, ceramides, sunscreens, especially ultraviolet screening agents, insect repellents, slimming agents, colorants, bactericides and antidandruff agents.

15. The use as claimed in any one of claims 5 to 14, characterized in that the composition is in the form of an aqueous, aqueous-alcoholic or oily solution, an oil-in-water or water-in-oil or multiple emulsion, an aqueous or oily gel, a liquid, pasty or solid anhydrous product or a dispersion of oil in an aqueous phase with the aid of spherules.

16. The use as claimed in any one of claims 5 to 15, characterized in that the composition has the appearance of a white or colored cream, an ointment, a milk, a lotion, a serum, a paste, a mousse or a solid.

17. The use as claimed in any one of claims 5 to 16, characterized in that the composition has a pH of between 3 and 8 and preferably between 5 and 7.

18. A cosmetic treatment process for protecting the body against the effects of pollution, characterized in that it consists in applying to keratin material a cosmetically effective amount of at least one derivative of formula (I) as defined in claim 1 or 4.

19. A cosmetic treatment process for protecting the body against the effects of pollution, characterized in that it consists in applying to keratin material a cosmetic composition as defined in any one of claims 5 to 17.
